(19) United States
(12) Patent Application Publication
(10) Pub. No.: US 2011/0159602 A1
(43) Pub. Date: Jun. 30, 2011

(54) ANALYSIS OF VI SACCHARIDES

(76) Inventors: Francesco Berti, Colle Val D'Elsa (IT); Francesca Micoli, Siena (IT); Daniela Proietti, Siena (IT)

(21) Appl. No.: 12/997,416

(22) PCT Filed: Jun. 12, 2009
(86) PCT No.: PCT/IB2009/006087

§ 371 Date: Feb. 25, 2011

(30) Foreign Application Priority Data
Jun. 13, 2008 (IT) MI2008A1079

(51) Int. Cl. G01N 33/00

(52) U.S. Cl. 436/94

(57) ABSTRACT

Salmonella typhi Vi saccharide can be assayed in two new ways. First, its proton NMR spectrum can be used, with comparison to an internal Standard permitting quantitative analysis. Second, anion exchange chromatography with amperometric detection can be used on hydrolysed saccharide.

This application claims the benefit of Italian patent application MI 2008 A 1079, filed 13 Jun. 2008, the complete contents of which are incorporated herein by reference.

### TECHNICAL FIELD

This invention is in the field of analysis of saccharides.

### BACKGROUND ART

Immunogens comprising capsular saccharide antigens conjugated to carrier proteins are well known in the art. Conjugation converts T-independent antigens into T-dependent antigens, thereby enhancing memory responses and allowing protective immunity to develop, and the prototype conjugate vaccine was for Haemophilus influenzae type b (Hib) [e.g. see chapter 14 of ref. 1]. Since the Hib vaccine, conjugated saccharide vaccines for protecting against Neisseria meningitidis (meningococcus) and against Streptococcus pneumoniae (pneumococcus) have been developed. Vaccines containing Vi, the capsular polysaccharide of Salmonella typhi (now referred to as Salmonella enterica serovar typhi), have also been shown to confer protection against typhoid fever in clinical trials [2,3], and a conjugate vaccine has also been prepared (e.g. [4]).

Where saccharides are included in vaccines and other biological products then regulatory authorities generally require their characterisation. However, Vi cannot be quantified by conventional colorimetric methods, and the standardisation of vaccines composed of conjugated or un-conjugated Vi has been hindered by this lack of a method for quantifying Vi [5]. Colorimetric methods for measuring amino sugars or uronic acids are not applicable to the measurement of Vi because its polyhexosaminuronic acid structure is resistant to acid hydrolysis and the aminouronic acid moieties do not form the chromophore in the carbazole assay.

Vi quantification assays based upon Fourier-transformed infrared spectroscopy of dried samples and spectrophotometric titration with acridine orange have been reported [5], as has a method for obtaining the molar ratios of O-acetyl to N-acetyl groups in Vi using high performance anion exchange chromatography (HPAEC) with conductivity detection (CD) [6]. Similarly, an NMR assay for identifying and measuring the O-acetyl content of Vi has been reported [7].

It is an object of the invention to provide further and improved methods and systems for quantifying Vi saccharide in a sample.

### DISCLOSURE OF THE INVENTION

The invention provides two simple and accurate approaches for quantifying Vi saccharide and its conjugates that allow the detection of very low (less than or equal to 5 μg/ml, including as low as 1 μg/ml) Vi concentrations. First, the proton NMR spectrum of de-O-acetylated Vi saccharide can be compared to an internal standard to determine the Vi saccharide concentration in a sample. The addition of known amounts of a reference compound to a NMR sample in order to determine the saccharide content has been reported [8]. However, the present approach of using a reference compound as an internal standard for a NMR assay of de-O-acetylated Vi saccharide is new. Second, liquid chromatography can be used on hydrolysed and de-acetylated Vi saccharide in order ascertain its concentration in a sample. This method is an improvement over prior art techniques, such as the acridine orange method referred to above, as it permits the quantification of Vi in a sample containing proteins, reagents, and other contaminants.

Accordingly, a first aspect of the invention provides a method for quantifying de-O-acetylated Vi saccharide in a sample by NMR spectroscopy. The Vi saccharide is fully de-O-acetylated. The N-acetyl group on the Vi saccharide is usually fully retained but, alternatively, a known proportion of the Vi saccharide may be de-N-acetylated. De-O-acetylation leaves a well resolved N-acetyl resonance that may be used in quantifying the Vi saccharide present.

The method comprises the steps of
i. de-O-acetylating any Vi saccharide present in the sample; and
ii. obtaining a NMR spectrum of the sample.

The NMR spectrum can then be used to calculate the amount of Vi saccharide present in the sample.

The method may further comprise the step of adding a known amount of a reference compound to the sample. Alternatively, the NMR instrument may have previously been calibrated using a known amount of a reference compound.

The calculation step may be performed by comparing one or more resonances from the NMR spectrum that are attributable to de-O-acetylated Vi saccharide to one or more resonances that are attributable to the reference compound.

A second aspect of the invention provides a method for quantifying Vi saccharide in a sample by liquid chromatography. Preferably, HPAEC is employed in this method of the invention. Prior to analysis by liquid chromatography, Vi saccharide in the sample is advantageously hydrolysed and de-acetylated. It is preferable if the Vi saccharide is hydrolysed and then de-acetylated. The hydrolysis step gives the formation of oligosaccharides, whilst the de-acetylation step completely de-acetylates these oligosaccharides and may further hydrolyse them to give the final product. Although de-N-acetylation facilitates subsequent hydrolysis, de-acetylation decreases the solubility in water of the saccharide, and so can give solubilisation problems if performed first. In some embodiments, though, a single step can achieve both results (e.g. base treatment can both hydrolyse and de-acetylate, whereas acid treatment may merely hydrolyse).

The method comprises the steps of:
i. hydrolysing Vi saccharide present in the sample;
ii. de-acetylating Vi saccharide present in the sample; and
iii. analysing the sample by liquid chromatography.

This aspect of the invention also provides a method for quantifying hydrolysed Vi saccharide in a sample. This method comprises the steps of:
i. de-acetylating hydrolysed Vi saccharide present in the sample; and
ii. analysing the sample by liquid chromatography.

This aspect of the invention also provides a method for quantifying de-acetylated Vi saccharide in a sample. This method comprises the steps of:
i. hydrolysing de-acetylated Vi saccharide present in the sample; and
ii. analysing the sample by liquid chromatography.

These methods may further comprise a second hydrolysis step that follows the de-acetylation step, but this is not always essential.

This aspect of the invention also provides a method for quantifying Vi saccharide in a sample, wherein the Vi saccharide has been hydrolysed and de-acetylated. This method comprises the step of: (i) analysing the sample by liquid chromatography.

The liquid chromatograph can then be used to calculate the amount of Vi saccharide present in the sample.

### Vi Saccharide

The methods of the invention are for analysing samples containing Vi. Vi is the capsular saccharide of Salmonella typhi (previously classified as a species itself, but now referred to as the typhi serovar of S. enterica), and may also be found in other serovars of Salmonella (such as S. enterica serovar paratyphi C or serovar dublin) and in other bacteria, such as Citrobacter (e.g. C. freundii and C. youngae). The methods of the present invention may be used to detect any Vi saccharide, regardless of its source.

Vi polysaccharide is a linear homopolymer of a hexosaminuronic acid, α1,4-N-acetylgalactosaminouronic acid, which is 60-90% O-acetylated at the C-3 position [9 to 14]. The O-acetyl substitution on a Vi saccharide is a factor in its ability to elicit a protective immune response [6]. This O-acetyl group is completely removed in the methods of the invention.

### NMR Spectroscopy

The method of the first aspect of the invention is preferably performed using ¹H NMR spectroscopy, although it is envisaged that spectra of other nuclei may be used, e.g. ¹³C NMR.

In this aspect of the invention, the Vi saccharide in a sample is fully de-O-acetylated. Full de-O-acetylation is required because the NMR peaks attributable to N-acetyl and O-acetyl in native Vi have the same chemical shift, and so if de-O-acetylation is incomplete then the integral of the N-acetyl peak cannot be accurately measured. It is preferable to use deuterated solvents and reagents, such as NaOD, KOD etc. in D₂O etc., for performing the de-O-acetylation as this reduces the concentration of water, which gives ¹H NMR resonances in the same region as carbohydrates, and thereby improves the quality of the spectrum. The alkaline medium that is given by the use of these reagents also gives a ¹H NMR spectrum in which the spectral dispersion is improved and the peaks are sharpened, thereby further improving its intelligibility. NaOD, if used, may be used at a concentration of from 50 to 1000 mM, or at a concentration of from 100 to 750 mM, or at a concentration of from 150 to 500 mM. A useful concentration of NaOD is 200 mM.

Whilst the saccharide is de-O-acetylated, a well resolved N-acetyl resonance remains. This N-acetyl resonance may be compared to an internal standard that is provided by adding a known amount of a reference compound to the test sample in order to ascertain the concentration of Vi saccharide present. Alternatively, the NMR instrument may have previously been calibrated using a known amount of the reference compound.

The reference compound should give a NMR spectrum that is distinct from that of the Vi saccharide, with no resonances having the same chemical shifts as the Vi saccharide. Useful reference compounds include citric acid and ethanol, citric acid (or a citrate salt) being particularly useful as it is available as a dry powder and therefore can easily be weighed to prepare a standard solution. The concentration of the reference compound that is used to calibrate an NMR instrument or that is present in a sample may be from 0.005 to 0.05 mM, for example from 0.0075 to 0.025 mM, or 0.01 mM.

### The Liquid Chromatography Column

The method of the second aspect of the invention may use various liquid chromatography columns, but preferably makes use of high performance liquid chromatography (HPLC). High performance anion exchange chromatography (HPAEC) is particularly preferred.

Useful columns are those that spontaneously retain saccharides such that the saccharides have to be eluted from the column. Elution from the chromatography column can be an isocratic elution or a gradient elution. Eluents including sodium hydroxide and/or sodium acetate are typical eluents used during HPAEC-PAD (pulsed amperometric detection) analysis of saccharides. Nitrate and/or chloride salt eluents (typically sodium salts) may also be used, usually substantially in the absence of any acetate eluent. For eluting analytes from anion exchange columns then the eluent will generally be basic e.g. the pH will be >8, >9, >10, >11, >12, >13, etc. Hydroxide salts (e.g. NaOH) can be used to achieve the desired pH, and hydroxide ions are typical for use in anion exchange eluents. A useful eluent is 40 to 150 mM NaNO₃ in NaOH 100 mM.

Eluates may be subjected to chemical suppression of hydroxide ions, particularly where the ions interfere with an analytical detection technique that is being used. A micromembrane suppressor can conveniently be used, such as the MMS products from Dionex™. The ‘MMS III’ product uses continuous chemical suppression to enhance analyte conductivities while decreasing eluent conductivity, and enables direct conductivity detection with ion-exchange applications using isocratic or gradient elution over wide concentration ranges. Suppressors that generate acetic acid from acetate ions may be avoided when acetate ions are included in the eluent and the generated acetic acid interferes with an analytical detection technique that is being used.

Useful HPAEC columns for use with the second aspect of the invention are the “CarboPac” columns marketed by Dionex, such as the PA1 [10 μm diameter polystyrene substrate 2% crosslinked with divinylbenzene, agglomerated with 500 nm MicroBead quaternary ammonium functionalized latex (5% crosslinked)], PA100, PA20, PA10 [10 μm diameter ethylvinylbenzene substrate 55% crosslinked with divinylbenzene, agglomerated with 460 nm MicroBead difunctional quaternary ammonium ion (5% crosslinked)], PA200 or MA1 columns.

A useful column is the CarboPac PA1 column, or the CarboPac PA10 column with a PA10 guard column. When used in its 4×250 mm analytical format, the PA10 column has a capacity of approximately 100 μeq (milliequivalents of charge).

### Amperometric Detection

The eluate of the liquid chromatography column is preferably analysed amperometrically in the second aspect of the invention, although other detection methods may also be employed.

The amperometric detection is preferably pulsed amperometric detection (PAD), as this does not require the chemical suppression of hydroxide ions. Various waveforms can be used in PAD [15]. A negative potential may be used for cleaning the electrode; this improves long term reproducibility and reduces electrode wear.

A useful electrode for the amperometric detection is a gold electrode.

As an alternative to using amperometric detection, the invention may use conductivity detection, in which the eluate may be subjected to chemical suppression of hydroxide ions.

### Analytes

The invention is useful for analysing Vi saccharide analytes. These may be polysaccharides (e.g. with a degree of polymerisation of at least 10, e.g. 20, 30, 40, 50, 60 or more) or oligosaccharides (e.g. with a degree of polymerisation of from 2 to 10). Oligosaccharides may be the result of depolymerisation and/or hydrolysis of a parent polysaccharide e.g. the analyte may be a saccharide-containing fragment of a larger saccharide. In addition to being useful for analysing full-length capsular saccharides, the methods of the invention can be used with oligosaccharide fragments of them.

In the method of the second aspect of the invention, the sample is treated to hydrolyse any Vi saccharide present. This hydrolysis can use acidic and/or basic conditions. The hydrolysis step may be carried out using trifluoroacetic acid (TFA), e.g. at a concentration of 4 M and at a temperature of 120° C. for 2 hours. The sample may then be treated with sodium hydroxide, e.g. at a concentration of 2 M and at a temperature of 110° C. for 6 hours in order to completely de-acetylate any Vi saccharide present. The hydrolysis step is preferably performed before the de-acetylation step, for the reasons discussed above. The Vi saccharide may then be further treated with TFA, e.g. at a concentration of 4 M and a temperature of 118° C. for 2 hours. Alkaline hydrolysis is ideal e.g. by NaOH treatment for at least 2 hours (e.g. >2 hours, >4 hours, etc) and can usefully achieve both hydrolysis and de-O-acetylation.

Vi saccharide antigens present in the analyte may be the product of cleavage from glycoconjugates e.g. from Vi saccharide-protein conjugate vaccine antigens.

The analyte may be a product to be tested prior to release (e.g. during manufacture or quality control testing), or may be a product to be tested after release (e.g. to assess stability, shelf-life, etc.).

### Analysis

Prior to addition of the sample to a liquid chromatography column in the second aspect of the invention, the sample is subjected to a hydrolysis step and a de-acetylation step, as discussed above. A characteristic peak is seen in the HPAEC-PAD analysis of a sample that has been treated in this way. HPAEC-PAD analyses of several such samples, each having different Vi saccharide concentrations (which may be determined using the method of the first aspect of the invention), show a linear trend for the integral of this peak. This result may be used to generate a calibration curve for a given instrument, allowing the subsequent quantification of Vi saccharide in a sample by a method that is less costly and requires less material than an NMR method.

Quantities of Vi saccharide can be determined in terms of numbers of molecules of Vi monosaccharide repeat units (e.g. moles), masses, ratios or concentrations. It is typical to work in moles in order to simplify the calculation of ratios, but any of these measures can be used and interchanged to determine saccharide content of a sample. For quantitative measurement, as noted above, analytical results may be compared to a standard with a known content of a particular compound.

### Further Steps

It may be desired to remove at least some non-analyte compounds from the sample before entry to the column in the method of the second aspect of the invention, and Dionex™ produce pre-column traps and guards for this purpose e.g. an amino trap for removing amino acids, a borate trap, etc.

After NMR analysis, or elution and analysis, the invention may include the further step of determining a characteristic of a detected analyte e.g. its purity, etc.

Again, after NMR analysis, or amperometric or conductivity detection, the sample or eluate may be coupled into a mass spectrometer e.g. FAB/MS or ESI/MS.

### Conjugates

The invention is useful for analysing Vi saccharide content of vaccines, and in particular for vaccines that comprise conjugated Vi saccharide. Covalent conjugation is used to enhance immunogenicity of saccharides by converting them from T-independent antigens to T-dependent antigens, thus allowing priming for immunological memory. The use of conjugation to carrier proteins in order to enhance the immunogenicity of carbohydrate antigens is well known [e.g. reviewed in refs. 16 to 24 etc.] and is used in particular for paediatric vaccines [25].

The carrier protein may be covalently conjugated to the Vi saccharide directly or via a linker.

Linkages via a linker group may be made using any known procedure, for example, the procedures described in references [26] and [27]. A typical type of linkage is an adipic acid linker, which may be formed by coupling a free —NH₂ group (e.g. introduced to a Vi saccharide by amination) with adipic acid (using, for example, diimide activation), and then coupling a protein to the resulting saccharide-adipic acid intermediate [20,28,29]. Preferably, Vi conjugates are prepared by a carbodiimide-mediated synthesis wherein adipic acid dihydrazide (ADH) [30] is used to derivatise the protein, and then the derivatised protein is bound to COOH groups of the Vi saccharide [31]. Alternatively, the protein can be derivatised with N-succinimidyl-3-(2-pyridyldithio)-propionate (SPDP), and the Vi saccharide can be derivatised with cystamine [32,33]. The protein and the Vi saccharide may then be covalently bound through disulfide exchange.

Yet another type of linkage is a carbonyl linker, which may be formed by reaction of a free hydroxyl group of a modified Vi saccharide with CDI [34,35] followed by reaction with a protein to form a carbamate linkage. Other linkers include 13-propionamido [36], nitrophenyl-ethylamine [37], haloacyl halides [38], glycosidic linkages [39], 6-aminocaproic acid [40], C₄ to C₁₂ moieties [41], etc. Carbodiimide condensation can also be used [42].

Typical carrier proteins are bacterial toxins, such as diphtheria or tetanus toxins, or toxoids or mutants thereof. These are commonly used in conjugate vaccines. The CRM₁₉₇ diphtheria toxin mutant is particularly useful [43].

Other carrier proteins include the N. meningitidis outer membrane protein complex [44], synthetic peptides [45,46], heat shock proteins [47,48], pertussis proteins [49,50], cytokines [51], lymphokines [51], hormones [51], growth factors [51], artificial proteins comprising multiple human CD4⁺ T cell epitopes from various pathogen-derived antigens [52] such as N19 [53], protein D from H. influenzae [54 to 56], pneumolysin [57] or its non-toxic derivatives [58], pneumococcal surface protein PspA [59], iron-uptake proteins [60], toxin A or B from C. difficile [61], recombinant Pseudomonas aeruginosa exoprotein A (rEPA) [62], etc. It is possible to use mixtures of carrier proteins. A single carrier protein may carry multiple Vi saccharides [63].

Conjugates may have excess carrier (w/w) or excess Vi saccharide (w/w) e.g. in the ratio range of 1:5 to 5:1, although broader ranges are also possible e.g. between 1:15 and 15:1. Conjugates with excess carrier protein are typical e.g. in the range 0.2:1 to 0.9:1, such as 0.5:1, or with equal weights (1:1). In some embodiments the Vi:protein ratio is between 0.4:1 and 1.2:1.

The invention is useful both before and after conjugation. In particular, after conjugation, compositions can be analysed using the invention in three ways: first, the Vi saccharide level in a composition can be measured e.g. prior to mixing of different conjugates, or prior to release of a vaccine (for regulatory or quality control purposes); second, the level of free, unconjugated Vi saccharide in a composition can be measured e.g. to check for incomplete conjugation, or to follow conjugate hydrolysis by monitoring increasing free saccharide over time; third, the level of conjugated Vi saccharide in a composition can be measured, for the same reasons. The first and third ways may involve the release of Vi saccharide from the conjugate prior to analysis.

The proportion of free, unconjugated Vi saccharide in a composition may be obtained by measuring the total amount of Vi saccharide in a sample of the composition and the amount of conjugated Vi saccharide in a sample of identical size and then subtracting the amount of conjugated Vi saccharide from the total amount of Vi saccharide and dividing by the total amount of Vi saccharide. Similarly, the proportion of conjugated Vi saccharide in a composition may be obtained by measuring the total amount of Vi saccharide in a sample of the composition and the amount of free, unconjugated Vi saccharide in a sample of identical size and then subtracting the amount of free, unconjugated Vi saccharide from the total amount of Vi saccharide and dividing by the total amount of Vi saccharide. The invention can be used in either way, and the skilled person can choose the most appropriate method at his own convenience.

To separately assess conjugated and unconjugated Vi saccharides, they must be separated. The conjugation reaction changes various chemical and physical parameters for the Vi saccharide, and the differences can be exploited for separation.

A method for analysing a glycoconjugate may comprise the steps of: (a) treating the glycoconjugate to release Vi saccharide from the carrier; and (b) analysing the released Vi saccharide as described above. The invention provides a method for releasing a vaccine for use by physicians, comprising the steps of: (a) manufacturing a vaccine comprising a conjugate of a Vi saccharide, (b) quantifying the Vi saccharide in the vaccine as described above; and, if the results from step (b) indicate a Vi saccharide level acceptable for clinical use, (c) releasing the vaccine for use by physicians. Step (b) may be performed on a packaged vaccine or on a bulk vaccine prior to packaging.

### Non-Saccharide Components

As well as analysing Vi saccharide in a composition, the process may include analysis of other components or properties e.g. osmolality, pH, degree of polymerisation for individual Vi saccharides or conjugates, protein content (particularly for carrier proteins), aluminium content, detergent content, preservative content, etc.

The invention provides a method for preparing a vaccine composition, comprising a step of analysing Vi saccharide as described above, and a step of pH measurement of the composition, optionally followed by a step of adjusting the pH of the composition to a desired value e.g. between 6 and 8, or about 7.

The invention also provides a method for preparing a vaccine composition, comprising the steps of: (a) providing Vi saccharide analysed as described above; (b) conjugating the Vi saccharide to one or more carrier proteins; (c) optionally, analysing the bulk vaccine for pH and/or other properties; and, if the results from step (c) indicate that the bulk vaccine is acceptable for clinical use, (d) preparing and packaging the vaccine for human use from the bulk. Step (c) may involve assessment of minimum saccharide concentration, assessment of unconjugated:conjugated saccharide ratio, etc. Step (d) may involve packaging into unit dose form or in multiple dose form e.g. into vials or into syringes. A typical human dose for injection has a volume of 0.5 ml.

The invention also provides a method for preparing a vaccine composition, comprising the steps of: (a) providing a sample of Vi saccharide analysed as described above; (b) conjugating the Vi saccharide to one or more carrier proteins, to give conjugated Vi saccharide; and (c) mixing the conjugated Vi saccharide with one or more further antigens. e.g. with an antigen from hepatitis A virus, such as inactivated virus [e.g. 64, 65]. Such antigens may be adsorbed to an aluminium salt adjuvant (e.g. a hydroxide or a phosphate).

### General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x means, for example, x±10%.

The methods of the invention can be used for analytical and/or preparative purposes. References to “analysing”, “analysis”, etc. should not be construed as excluding preparative methods.

“Vi saccharide” will be understood to refer to Vi, the capsular saccharide of Salmonella typhi and Citrobacter freundii, but may also refer to any structurally or antigenically identical saccharides e.g. pectin N-acetylated at C-2 and O-acetylated at C-3.

Unless specifically stated, a process comprising a step of mixing two or more components does not require any specific order of mixing. Thus components can be mixed in any order. Where there are three components then two components can be combined with each other, and then the combination may be combined with the third component, etc.

The term “quantifying” encompasses both measuring the amount of substance in a sample precisely and comparatively i.e. determining whether it lies above or below a threshold value.

### BRIEF DESCRIPTION OF DRAWINGS

FIG. 1 shows a depiction of the structural formula of Salmonella typhi Vi (α1,4-N-acetylgalactosaminouronic acid).

FIGS. 2 and 3 show ¹H NMR spectra of native Vi saccharide and de-O-acetylated Vi saccharide respectively. X indicates unresolved peaks of N-acetyl and O-acetyl; Y indicates acetate anion (present in native Vi saccharide in very small quantities); Z indicates N-acetyl.

FIG. 4 shows a ¹H NMR spectrum of de-O-acetylated Vi saccharide in the presence of ethanol as a reference compound. b: acetate anion; c: N-acetyl; d: H-1; e: H-2; f: H-3; g: H-4; h: H-5.

FIG. 5 shows a ¹H NMR spectrum of citric acid.

FIG. 6 shows part of the ¹H NMR spectrum of a sample of de-O-acetylated Vi saccharide in the presence of citric acid as a reference compound. Brackets indicate citrate and N-Ac.

FIG. 7 shows a HPAEC-PAD analysis of Vi saccharide after hydrolysis with TFA at a concentration of 4 M and at a temperature of 120° C. for 2 hours.

FIG. 8 shows a HPAEC-PAD analysis of Vi saccharide after i) de-O-acetylation and ii) hydrolysis with TFA at a concentration of 4 M and at a temperature of 120° C. for 2 hours.

FIG. 9 shows a HPAEC-PAD analysis of Vi saccharide after i) complete de-acetylation with sodium hydroxide at a concentration of 2 M and at a temperature of 110° C. for 6 hours and ii) hydrolysis with TFA at a concentration of 4 M and at a temperature of 120° C. for 2 hours.

FIG. 10 shows a HPAEC-PAD analysis of Vi saccharide after hydrolysis with TFA at a concentration of 4 M and at a temperature of 120° C. for 2 hours and ii) complete de-acetylation with sodium hydroxide at a concentration of 2 M and at a temperature of 110° C. for 6 hours.

FIG. 11 shows a calibration curve generated by plotting the integral of the peak marked by the arrow in FIG. 10 against Vi concentration (determined by ¹H NMR; 5-200 m/ml). R²=0.997.

FIG. 12 shows a HPAEC-PAD analysis of a Vi saccharide conjugate, following hydrolysis with TFA at a concentration of 4 M and at a temperature of 120° C. for 2 hours and complete de-acetylation with 2M sodium hydroxide at 110° C. for 6 hours.

FIG. 13 shows a further calibration curve from 0-200 μg/ml Vi. R²=0.9993.

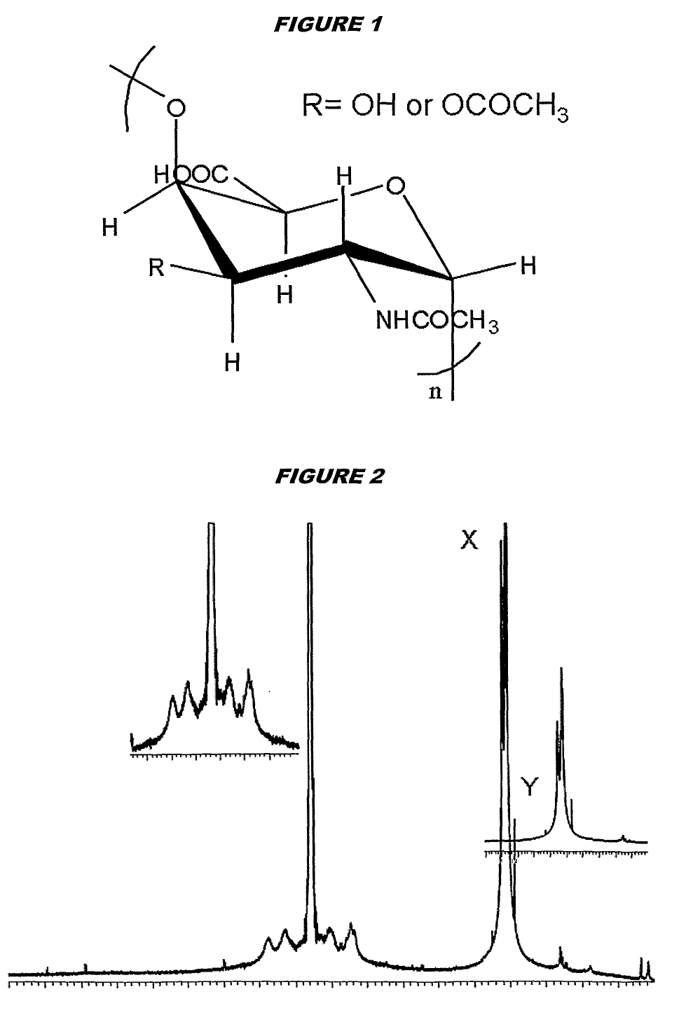

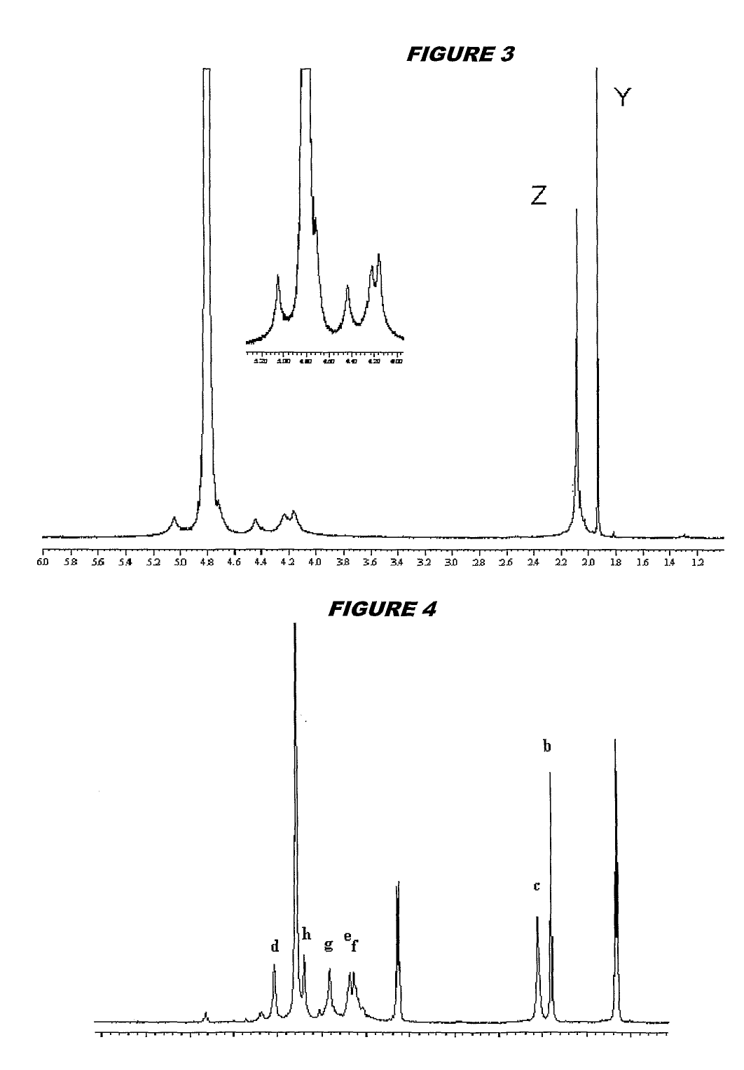

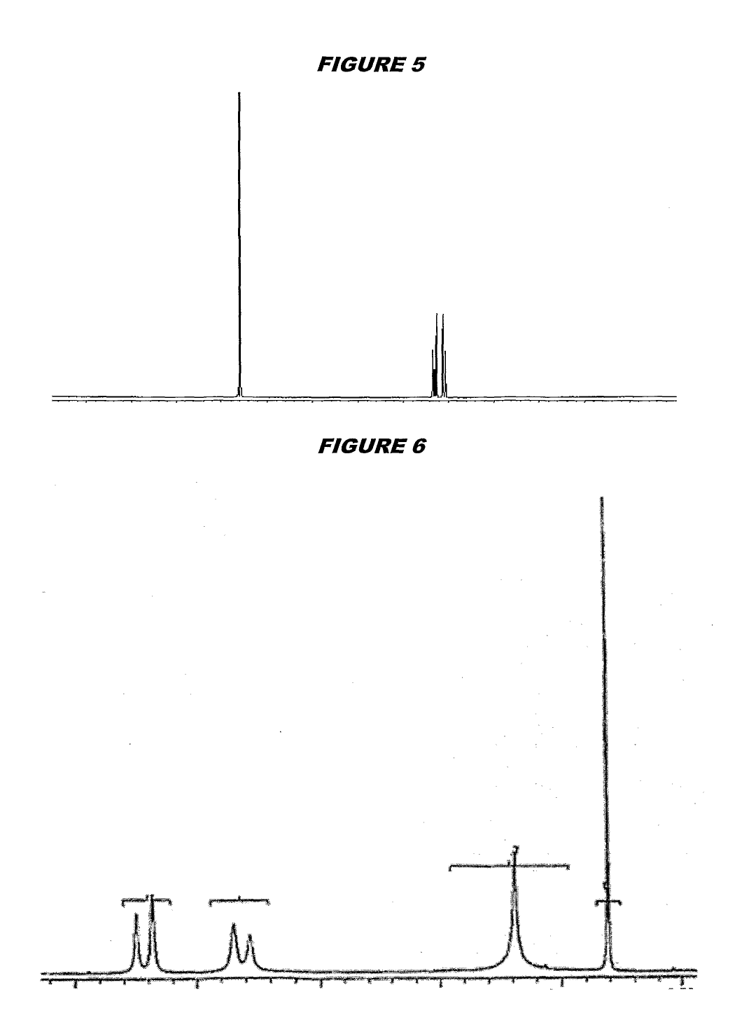

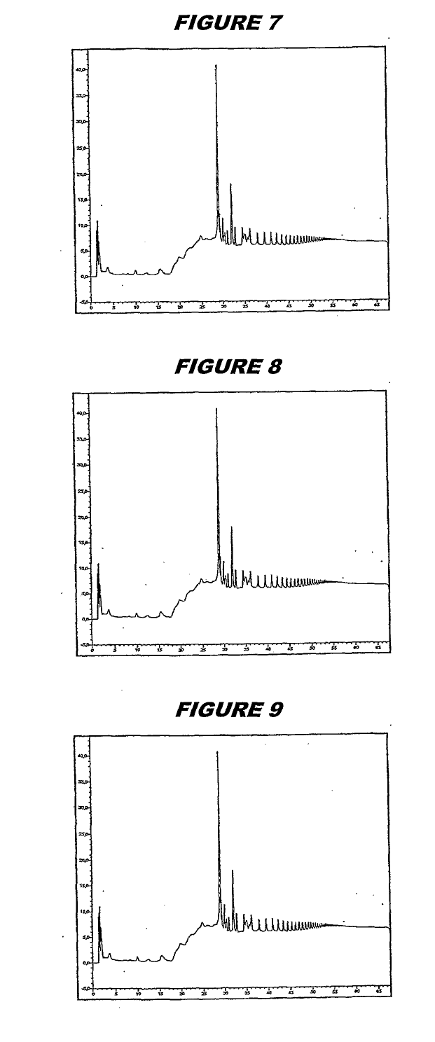

### MODES FOR CARRYING OUT THE INVENTION

### NMR Analysis

15 μl of a standard solution of citrate (0.4328 mmol/ml, prepared by dissolving 127.3 mg of trisodium citrate (294.1 gmol⁻¹) in 1 ml of D₂O) was added to a solution of (nominally, according to the dry weight of the Vi saccharide powder) 0.015 mmol of Vi saccharide in NaOD (200 mM). The NMR spectrum shown in FIG. 6 was obtained. The purity of the Vi saccharide powder was calculated as follows:

Integral of the resonance from NCOCH₃ (3H): 92; 92/3=30.66 (integral corresponding to 1H)

Integral of the resonance from citrate (4H): 100; 100/4=25 (integral corresponding to 1H)

Molar ratio of Vi saccharide/citrate from the spectrum=30.66/25=1.2264

No. of moles of citrate added in the tube: 0.0065 mmol

No. of moles Vi saccharide therefore=0.0065×1.2264=0.008 mmol

As noted above, the no. of moles of Vi saccharide in the solution according to the dry weight of the Vi saccharide powder: 0.015 mmol

The purity of the Vi saccharide powder=0.008/0.015×100=53.3%

Spectral assignment of ¹H NMR de-O-acetylated Vi saccharide in the presence of citrate or ethanol standards (RT, 500 MHz, in D₂O):

5.10 ppm H1, 4.70 ppm H5, 4.44 ppm H4, 4.2 ppm H2, 4.13 ppm H3, 2.06 ppm N-acetyl in non-O-acetylated residues (3H), 1.91 ppm acetate anion arising during de-O-acetylation (3H), 2.72-2.48 ppm 4H citrate, 3.62 ppm (2H, CH₃CH₂OH), 1.25 ppm (3H, CH₃CH₂OH).

The parameters of the instrument (a Bruker DRX 500 MHz NMR spectrometer) used to obtain the spectra listed above were as follows:
Nucleus ¹H
NS Number of scans 128
Number of data points TD 32768
Number of dummy scans DS 0
Spectral width Hz SWH 5000
Acquisition time AQ [sec] 3.2768500
Receiver gain adjust RGA 256
Dwell time DW 100.00 μs
Pre scan-delay D 198.86 μs
D1 relaxation delay [sec] 10 (>5*T1)
Dimension of accumulation loop TDO 1
Channel f1
P1 [μs]4.00, f1 channel high power pulse
PL1 [dB] 0.00, f1 channel power level for pulse

### HPAEC-PAD Analysis

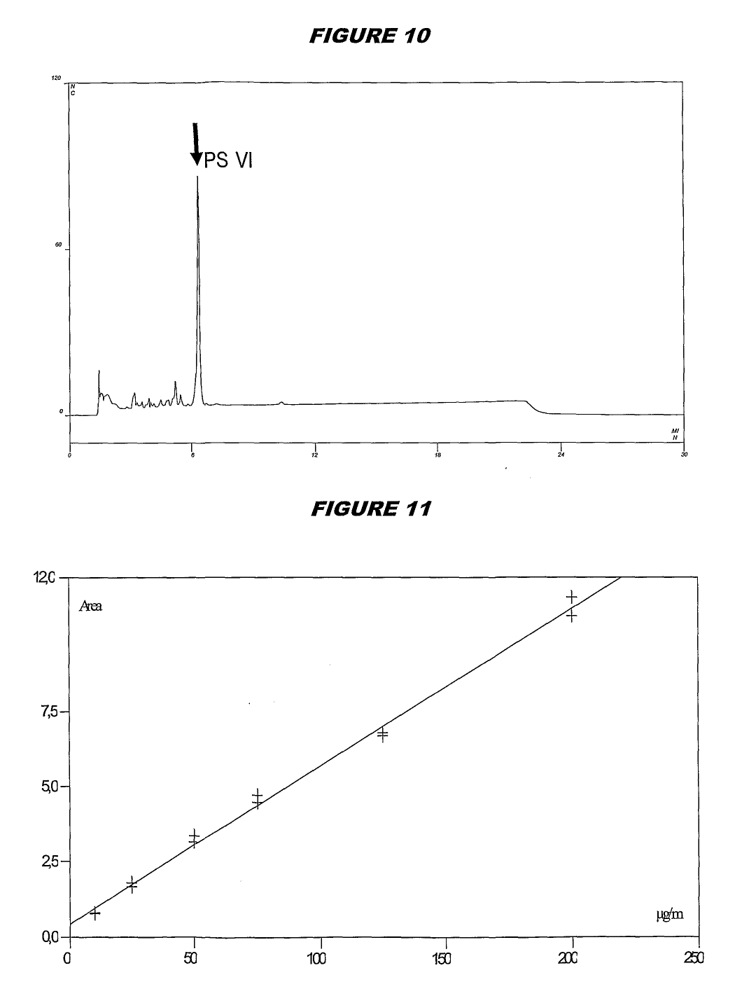

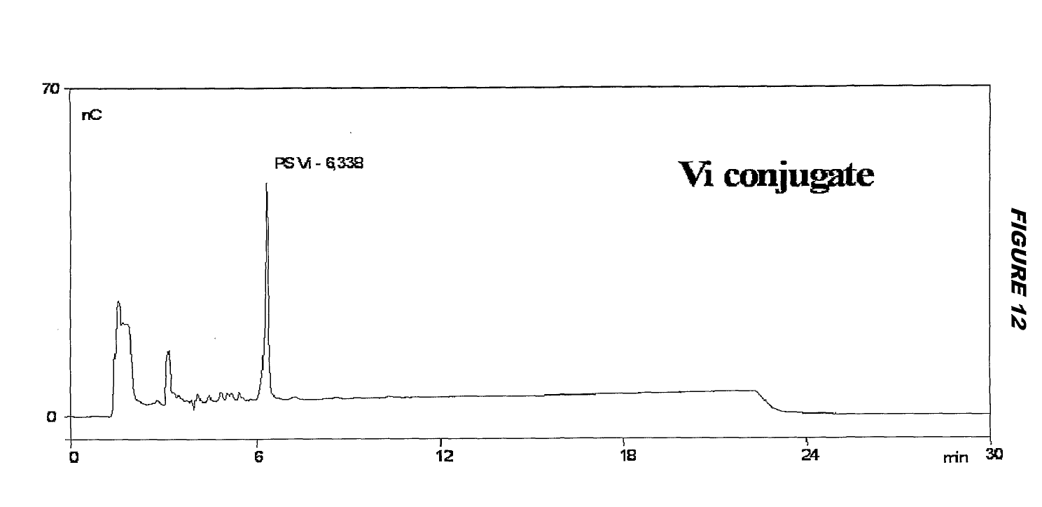

Treatment of a sample containing Vi saccharide with:
TFA 4 M, 2 hours, 120° C.
NaOH 2 M, 6 hours, 110° C.
   yielded the peak that is indicated by the arrow in FIG. 10. Samples with Vi concentrations ranging from 5 to 200 μg/ml (determined by ¹H NMR) treated in the same way as above showed a linear trend for the indicated integration peak detected by HPAEC-PAD analyses (see FIG. 11). This calibration curve allows Vi quantification to be carried out.

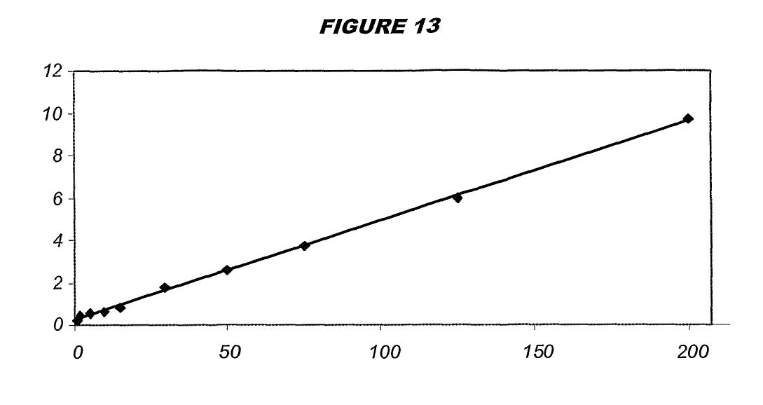

Further experiments confirmed that the method can detect saccharides at concentrations even as low as 1 μg/ml. FIG. 13 is a calibration curve from such experiments.

Complete hydrolysis was also achieved by using NaOH for 4 hours, thereby avoiding the need for the extra TFA step and reducing the NaOH treatment by 2 hours. A shorter NaOH treatment was also effective (2 hours) but gave lower peak areas.

The instrument used was a CarboPac PA10 column with PA10 guard-column. 40 mM NaNO₃ in NaOH 100 mM was used as the eluent.

It will be understood that the invention has been described by way of example only and modifications may be made whilst remaining within the scope and spirit of the invention.

### REFERENCES

### The Contents of which are Hereby Incorporated by Reference

[1] Vaccines (eds. Plotkin et al.) 4th edition, ISBN: 0721696880.
[2] Acharya et al. (1987) N Engl J Med 317:1101-1104.
[3] Klugman et al. (1987) Lancet ii:1165-1169.
[4] Canh et al. (2004) Infect Immun. 72(11):6586-8.
[5] Stone et al. (1988) J Clin Microbiol 26(4):719-25.
[6] Kao et al. (2004) Vaccine 22:335-344.
[7] Lernercinier et al. (2000) Biologicals 28(1):17-24.
[8] Jones (2005) J Pharm Biomed Anal 38(5):840-50.
[9] Clark et al. (1958) J Biol Chem 230:81-89.
[10] Heyns et al. (1967) Carbohydr Res 3:340-353.
[11] Heyns et al. (1959) Chem Ber 92:2435-2438.
[12] Webster et al. (1954) Arch Biochem Biophys 50:223-224.
[13] Webster et al. (1954) J Immunol 73:16-22.
[14] Whiteside et al. (1961) J Immunol 86:538-542.
[15] LaCourse & Johnson (1993) Anal Chem 65:50-55.
[16] Lindberg (1999) Vaccine 17 Suppl 2:S28-36.
[17] Buttery & Moxon (2000) J R Coll Physicians Lond 34:163-8.
[18] Ahmad & Chapnick (1999) Infect Dis Clin North Am 13:113-33, vii.
[19] Goldblatt (1998) J. Med. Microbiol. 47:563-567.
[20] EP-B-0 477 508.
[21] U.S. Pat. No. 5,306,492.
[22] WO98/42721.
[23] Dick et al. in Conjugate Vaccines (eds. Cruse et al.) Karger, Basel, 1989, Vol. 10, 48-114.
[24] Hermanson Bioconjugate Techniques, Academic Press, San Diego Calif. (1996).
[25] Ramsay et al. (2001) Lancet 357(9251):195-6.
[26] U.S. Pat. No. 4,882,317.
[27] U.S. Pat. No. 4,695,624.
[28] Mol. Immunol., 1985, 22, 907-919.
[29] EP-A-0208375.
[30] U.S. Pat. No. 4,965,338.
[31] Kossaczka et al. (1997) Infect Immun 65(6):2088-93.
[32] U.S. Pat. No. 5,204,098.
[33] Szu et al. (1989) Infect Immun 57(12):3823.
[34] Bethell G. S. et al., J. Biol. Chem., 1979, 254, 2572-4.
[35] Hearn M. T. W., J. Chromatogr., 1981, 218, 509-18.
[36] WO00/10599.
[37] Gever et al., Med. Microbiol. Immunol, 165: 171-288 (1979).
[38] U.S. Pat. No. 4,057,685.
[39] U.S. Pat. Nos. 4,673,574; 4,761,283; 4,808,700.
[40] U.S. Pat. No. 4,459,286.
[41] U.S. Pat. No. 4,663,160.
[42] WO2007/000343.
[43] Research Disclosure, 453077 (January 2002).
[44] EP-A-0372501.
[45] EP-A-0378881.
[46] EP-A-0427347.
[47] WO93/17712.
[48] WO94/03208.
[49] WO98/58668.
[50] EP-A-0471177.
[51] WO91/01146.
[52] Falugi et al. (2001) Eur J Immunol 31:3816-3824.
[53] Baraldo et al. (2004) Infect Immun 72(8):4884-7.
[54] EP-A-0594610.
[55] Ruan et al. (1990) J Immunol 145:3379-3384.
[56] WO00/56360.
[57] Kuo et al. (1995) Infect Immun 63:2706-13.
[58] Michon et al. (1998) Vaccine. 16:1732-41.
[59] WO02/091998.
[60] WO01/72337.
[61] WO00/61761.
[62] WO00/33882.
[63] WO99/42130.
[64] Bell (2000) Pediatr Infect Dis J 19:1187-1188.
[65] Iwarson (1995) APMIS 103:321-326.

1. A method for quantifying Vi saccharide present in a sample, comprising steps the of:
i. de-O-acetylating any Vi saccharide present in the sample; and
ii. obtaining a NMR spectrum of the sample.

2. The method of claim 1, further comprising the step of: (iii) using the NMR spectrum to calculate the amount of Vi saccharide present in the sample.

3. The method of claim 1, further comprising the step of: (iv) adding a known amount of a reference compound to the sample.

4. The method of claim 1, wherein an N-acetyl resonance is used to calculate the amount of Vi saccharide present in the sample.

5. The method of claim 1, wherein the NMR spectroscopy is ¹H NMR spectroscopy.

6. The method of claim 1, wherein the Vi saccharide is de-O-acetylated by sodium deuteroxide.

7. The method of claim 2, wherein the reference compound is selected from the group consisting of citric acid and ethanol.

8. A method for quantifying Vi saccharide present in a sample comprising performing liquid chromatography on said sample.

9. The method of claim 8, wherein the liquid chromatography is high performance anion exchange chromatography (HPAEC).

10. The method of claim 9, wherein pulsed amperometric detection (PAD) is used (HPAEC-PAD).

11. The method of claim 8, comprising steps of: (i) hydrolysing Vi saccharide present in the sample; (ii) de-acetylating Vi saccharide present in the sample; and (iii) analysing the sample by liquid chromatography.

12. The method of claim 11, wherein the method further comprises a second hydrolysis step that follows the de-acetylation step

13. The method of claim 11, wherein the hydrolysis step is carried out by treatment with trifluoroacetic acid (TFA) at a concentration of 4 M and at a temperature of 120° C. for 2 hours.

14. The method of claim 11, wherein the de-acetylation step is carried out by treatment with sodium hydroxide at a concentration of 2 M and at a temperature of 110° C. for 6 hours.

15. The method of claim 11, wherein hydrolysis and de-acetylation involves treatment with sodium hydroxide at a temperature of 100-150° C. for 2 to 6 hours.

16. The method of claim 1, wherein the Vi saccharide is from Salmonella typhi.

17. The method of claim 1, wherein the Vi saccharide is from Citrobacter freundii.

18. The method of claim 12, wherein the hydrolysis step(s) is carried out by treatment with trifluoroacetic acid (TFA) at a concentration of 4 M and at a temperature of 120° C. for 2 hours.

19. The method of claim 12, wherein hydrolysis and de-acetylation involves treatment with sodium hydroxide at a temperature of 100-150° C. for 2 to 6 hours.
